(19) United States
(12) Patent Application Publication
(10) Pub. No.: US 2025/0099667 A1
(43) Pub. Date: Mar. 27, 2025

(54) SUCTION INSTRUMENT WITH DEFORMABLE MULTI-PIECE GRIP ASSEMBLY

(71) Applicant: Acclarent, Inc., Irvine, CA (US)

(72) Inventors: Cesar FUENTES-ORTEGA, Pasadena, CA (US); George GUSEIN, Acre (IL); Raymond Yue-sing TANG, Rosemead, CA (US); Henry F. SALAZAR, Pico Rivera, CA (US); Alex GONZALEZ, Lake Forest, CA (US); Sheila Ruiz, Mission Viejo, CA (US); Hao H. TRAN, Santa Ana, CA (US); Priyadarshini GHOSH, Rancho Mission Viejo, CA (US)

(21) Appl. No.: 18/895,368

(22) Filed: Sep. 24, 2024

Related U.S. Application Data
(60) Provisional application No. 63/540,129, filed on Sep. 25, 2023.

(51) Int. Cl. A61M 1/00; A61M 39/10

(52) U.S. Cl. CPC A61M 1/741; A61M 1/76; A61M 39/10; A61M 2039/1027; A61M 2039/1077; A61M 2205/0216; A61M 2205/3334; A61M 2205/50; A61M 2209/00

(57) ABSTRACT

During surgery, debris being cleared from the target anatomy may inadvertently inhibit fluid communication between a suction instrument and a suction source. Therefore, it may be desirable to facilitate access to interior portions of the suction instrument and control of the amount of suction being provided through the suction instrument. The suction instrument can include an elongate cannula with a distal portion configured to be positioned within or adjacent to an anatomical passageway of a patient. The elongate cannula can define a suction lumen that can provide suction at the distal portion. A handle assembly can be associated with the elongate cannula and include a deformable grasping body defining a fluid communication chamber that can provide suction to the suction lumen. The deformable grasping body can elastically deform between relaxed and compressed states to alter the fluid communication between the fluid communication chamber and the suction lumen.

### CROSS-REFERENCE TO RELATED APPLICATIONS

This application claims the benefit of U.S. Provisional Application No. 63/540,129, filed Sep. 25, 2023, the entirety of which is incorporated herein by reference.

### BACKGROUND

In some instances, it may be desirable to operate within or adjacent to an anatomical passageway of a patient, such as performing an incision of mucosa, removal of bone, or dilation of an anatomical passageway. Such operations may occur within anatomical passageways such as ostia of paranasal sinuses (e.g., to treat sinusitis), the larynx, the Eustachian tube, or other passageways within the car, nose, or throat, etc. In addition to the above-described operations, or similar operations, it may be desirable to apply suction and/or irrigation within or adjacent to an anatomical passageway before, during, or after the above-described operations, or similar operations. One method of applying suction within or adjacent to an anatomical passageway of a patient involves obtaining a suction device having an elongate shaft defining a lumen terminating at an open distal end of the elongate shaft, where the lumen is in fluid communication with an external suction source. An operator may then insert the distal end of the elongate shaft within the nostril or mouth of a patient toward a desired location within the patient. With the distal end of the elongate shaft inserted within the patient, an operator may manipulate the suction device and/or suction source in order to remove extraneous and/or undesired matter near or within an anatomical passageway of a patient. Applying suction and/or irrigation during an operation may be beneficial for multiple purposes as will be apparent to those skilled in the art.

Image-guided surgery (IGS) is a technique where a computer is used to obtain a real-time correlation of the location of an instrument that has been inserted into a patient's body to a set of preoperatively obtained images (e.g., a CT or MRI scan, 3-D map, etc.), such that the computer system may superimpose the current location of the instrument on the preoperatively obtained images. An example of an electromagnetic IGS navigation system that may be used in IGS procedures is the TRUDI® Navigation System by Acclarent, Inc., of Irvine, California. In some IGS procedures, a digital tomographic scan (e.g., CT or MRI, 3-D map, etc.) of the operative field is obtained prior to surgery. A specially programmed computer is then used to convert the digital tomographic scan data into a digital map. During surgery, some instruments can include sensors (e.g., electromagnetic coils that emit electromagnetic fields and/or are responsive to externally generated electromagnetic fields), which can be used to perform the procedure while the sensors send data to the computer indicating the current position of each sensor-equipped instrument. The computer correlates the data it receives from the sensors with the digital map that was created from the preoperative tomographic scan. The tomographic scan images are displayed on a video monitor along with an indicator (e.g., crosshairs or an illuminated dot, etc.) showing the real-time position of each surgical instrument relative to the anatomical structures shown in the scan images. The surgeon is thus able to know the precise position of each sensor-equipped instrument by viewing the video monitor even if the surgeon is unable to directly visualize the instrument itself at its current location within the body.

It may be desirable to provide features that further facilitate the use of a suction device in ENT procedures and other medical procedures and, in some instances, in conjunction with an IGS navigation system. While several systems and methods have been made and used with respect to IGS and ENT surgery, it is believed that no one prior to the inventors has made or used the inventions described in the appended claims.

### BRIEF DESCRIPTION OF THE DRAWINGS

The accompanying drawings, which are incorporated in and constitute a part of this specification, illustrate embodiments of the inventions, and, together with the general description of the inventions given above, and the detailed description of the embodiments given below, serve to explain the principles of the present inventions.

FIG. 1 depicts a schematic view of an example of a IGS navigation system being used on a patient seated in an example of a medical procedure chair, in accordance with some embodiments.

FIG. 2 depicts a perspective view of an example of a suction instrument that may be used in conjunction with the navigation system of FIG. 1, in accordance with some embodiments.

FIG. 3 depicts a perspective view of a removable fluid coupling body of a handle assembly of the suction instrument of FIG. 2, in accordance with some embodiments.

FIG. 4 depicts a perspective view of a grasping portion of a handle assembly of the suction instrument of FIG. 2, in accordance with some embodiments.

FIG. 5A depicts a perspective view of the removable fluid coupling body of FIG. 3 and the grasping portion of FIG. 4 detached from each other, yet aligned with each other in preparation for attachment, in accordance with some embodiments.

FIG. 5B depicts a perspective view of the removable fluid coupling body of FIG. 3 initially inserted into the grasping portion of FIG. 4, in accordance with some embodiments.

FIG. 5C depicts a perspective view of the removable fluid coupling body of FIG. 3 coupled to the grasping portion of FIG. 4, in accordance with some embodiments.

FIG. 6A depicts a cross-sectional view of the handle assembly of the suction instrument of FIG. 2, taken along line 6-6 of FIG. 2, in accordance with some embodiments.

FIG. 6B depicts a cross-sectional view of the handle assembly of the suction instrument of FIG. 2, taken along line 6-6 of FIG. 2, with the grasping portion of FIG. 4 in a compressed state, in accordance with some embodiments.

FIG. 7 depicts a perspective view of another example of a suction instrument that may be used in conjunction with the navigation system of FIG. 1, in accordance with some embodiments.

FIG. 8 depicts an exploded perspective view of a proximal portion of the suction instrument of FIG. 7, showing a reusable fluid coupling body, a disposable shaft assembly, and a reusable cable detached from each other, in accordance with some embodiments.

FIG. 9 depicts a perspective view of the disposable shaft assembly of FIG. 8

FIG. 10 depicts a top perspective view of the reusable fluid coupling body of FIG. 8, in accordance with some embodiments.

FIG. 11 depicts bottom perspective view of the reusable fluid coupling body of FIG. 8, in accordance with some embodiments.

FIG. 12 depicts a top perspective view of the reusable cable of FIG. 8, in accordance with some embodiments.

FIG. 13 depicts a bottom perspective view of the reusable cable of FIG. 8, in accordance with some embodiments.

FIG. 14A depicts a bottom perspective view of the reusable fluid coupling body of FIG. 8 detached from the reusable cable of FIG. 8, in accordance with some embodiments.

FIG. 14B depicts a bottom perspective view of the reusable fluid coupling body of FIG. 8 attached to the reusable cable of FIG. 8, in accordance with some embodiments.

FIG. 15A depicts a top perspective view of the reusable fluid coupling body of FIG. 8 attached to the reusable cable of FIG. 8, yet detached from the disposable shaft assembly of FIG. 8, in accordance with some embodiments.

FIG. 15B depicts a top perspective view of the reusable fluid coupling body of FIG. 8, the reusable cable of FIG. 8, and the disposable shaft assembly of FIG. 8 attached to each other to form the suction instrument of FIG. 7, in accordance with some embodiments.

The drawings are not intended to be limiting in any way, and it is contemplated that various embodiments of the inventions may be carried out in a variety of other ways, including those not necessarily depicted in the drawings. The accompanying drawings incorporated in and forming a part of the specification illustrate several aspects of the present inventions, and together with the description serve to explain the principles of the inventions. It being understood, however, that these inventions are not limited to the precise arrangements shown.

### DETAILED DESCRIPTION

The following description of certain examples of the inventions should not be used to limit the scope of the present inventions. Other examples, features, aspects, embodiments, and advantages of the inventions will become apparent to those skilled in the art from the following description, which is by way of illustration, one of the best modes contemplated for carrying out the inventions. As will be realized, the inventions are capable of other different and obvious aspects, all without departing from the inventions. Accordingly, the drawings and descriptions should be regarded as illustrative in nature and not restrictive.

For clarity of disclosure, the terms “proximal” and “distal” are defined herein relative to a surgeon, or other operator, grasping a surgical instrument having a distal surgical end effector. The term “proximal” refers to the position of an element arranged closer to the surgeon, and the term “distal” refers to the position of an element arranged closer to the surgical end effector of the surgical instrument and further away from the surgeon. Moreover, to the extent that spatial terms such as “top,” “bottom,” “upper,” “lower,” “vertical,” “horizontal,” or the like are used herein with reference to the drawings, it will be appreciated that such terms are used for illustrative description purposes only and are not intended to be limiting or absolute. In that regard, it will be understood that surgical instruments such as those disclosed herein may be used in a variety of orientations and positions not limited to those shown and described herein.

As used herein, the terms “about” and “approximately” for any numerical values or ranges are intended to encompass the exact value(s) referenced as well as a suitable dimensional tolerance that allows the part or collection of components to function for its intended purpose as described herein.

### I. Example of an IGS Navigation System

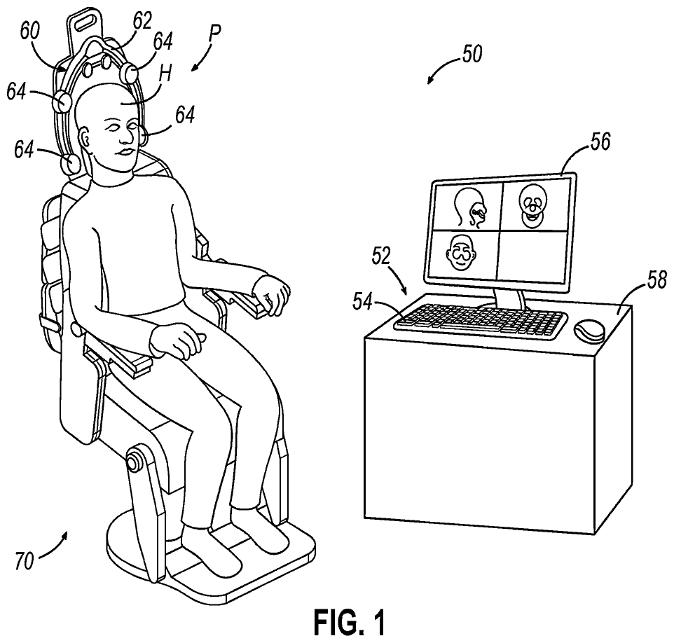

When performing a medical procedure within a head of a patient (P), it may be desirable to have information regarding the position of an instrument within the head (H) of the patient (P), particularly when the instrument is in a location where it is difficult or impossible to obtain an endoscopic view of a working element of the instrument within the head of the patient (P). FIG. 1 shows an example of an IGS navigation system 50 enabling a medical procedure to be performed within a head (H) of a patient (P) using image guidance. In addition to or in lieu of having the components and operability described herein The IGS navigation system 50 may be constructed and operable in accordance with at least some of the teachings of U.S. Pat. No. 7,720,521, entitled “Methods and Devices for Performing Procedures within the Ear, Nose, Throat and Paranasal Sinuses,” issued May 18, 2010; and/or U.S. Pat. No. 11,065,061, entitled “Systems and Methods for Performing Image Guided Procedures within the Ear, Nose, Throat and Paranasal Sinuses,” issued Jul. 20, 2021. The disclosures of U.S. Pat. No. 7,720,521, entitled “Methods and Devices for Performing Procedures within the Ear, Nose, Throat and Paranasal Sinuses,” issued May 18, 2010, and U.S. Pat. No. 11,065,061, entitled “Systems and Methods for Performing Image Guided Procedures within the Ear, Nose, Throat and Paranasal Sinuses,” issued Jul. 20, 2021, are incorporated by reference herein, in their entireties.

The IGS navigation system 50 of the present example comprises a field generator assembly 60, which comprises a set of magnetic field generators 64 that are integrated into a horseshoe-shaped frame 62. The field generators 64 are operable to generate alternating magnetic fields of different frequencies around the head (H) of the patient (P). An instrument may be inserted into the head (H) of the patient (P). Such an instrument may include one or more position sensors, as described in greater detail below. In the present example, the frame 62 is mounted to a chair 70, with the patient (P) being seated in the chair 70 such that the frame 62 is located adjacent to the head (H) of the patient (P). By way of example only, the chair 70 and/or the field generator assembly 60 may be configured and operable in accordance with at least some of the teachings of U.S. Pat. No. 10,561,370, entitled “Apparatus to Secure Field Generating Device to Chair,” issued Feb. 18, 2020. The disclosure of U.S. Pat. No. 10,561,370, entitled “Apparatus to Secure Field Generating Device to Chair,” issued Feb. 18, 2020, is incorporated by reference herein in its entirety. In some other variations, the patient (P) lies on a table, and the field generator assembly 60 is positioned on or near the table.

The IGS navigation system 50 of the present example further comprises a processor 52, which controls the field generators 64 and other elements of the IGS navigation system 50. For instance, the processor 52 is operable to drive the field generators 64 to generate alternating electromagnetic fields and process signals from the instrument to determine the location of a position sensor in the instrument within the head (H) of the patient (P). The processor 52 comprises a processing unit (e.g., a set of electronic circuits arranged to evaluate and execute software instructions using combinational logic circuitry or other similar circuitry) communicating with one or more memories. The processor 52 of the present example is mounted in a console 58, which comprises the operating controls 54 that include a keypad and/or a pointing device such as a mouse or trackball. A physician uses the operating controls 54 to interact with the processor 52 while performing the surgical procedure.

While not shown, the instrument may include a position sensor that is responsive to positioning within the alternating magnetic fields generated by the field generators 64. A coupling unit (not shown) may be secured to the proximal end of the instrument and may be configured to provide communication of data and other signals between the console 58 and the instrument. The coupling unit may provide wired or wireless communication of data and other signals.

In some versions, the position sensor of the instrument may comprise at least one coil at or near the distal end of the instrument. When such a coil is positioned within an alternating electromagnetic field generated by the field generators 64, the alternating magnetic field may generate electrical current in the coil, and this electrical current may be communicated along the electrical conduit(s) in the instrument and further to the processor 52 via the coupling unit. This phenomenon may enable the IGS navigation system 50 to determine the location of the distal end of the instrument within a three-dimensional space (i.e., within the head (H) of the patient (P), etc.). To accomplish this, the processor 52 executes an algorithm to calculate location coordinates of the distal end of the instrument from the position-related signals of the coil(s) in the instrument. Thus, a navigation sensor may serve as a position sensor by generating signals indicating the real-time position of the sensor within a three-dimensional space.

The processor 52 uses software stored in a memory of the processor 52 to calibrate and operate the IGS navigation system 50. Such operation includes driving the field generators 64, processing data from the instrument, processing data from the operating controls 54, and driving the display screen 56. In some implementations, operation may also include monitoring and enforcement of one or more safety features or functions of the IGS navigation system 50. The processor 52 is further operable to provide video in real-time via the display screen 56, showing the position of the distal end of the instrument in relation to a video camera image of the patient's head (H), a CT scan image of the patient's head (H), and/or a computer-generated, three-dimensional model of the anatomy within and adjacent to the patient's nasal cavity. The display screen 56 may display such images simultaneously and/or superimposed on each other during the surgical procedure. Such displayed images may also include graphical representations of instruments that are inserted in the patient's head (H), such that the operator may view the virtual rendering of the instrument at its actual location in real-time. By way of example only, the display screen 56 may provide images in accordance with at least some of the teachings of U.S. Pat. No. 10,463,242, entitled “Guidewire Navigation for Sinuplasty,” issued Nov. 5, 2019. The disclosure of U.S. Pat. No. 10,463,242, entitled “Guidewire Navigation for Sinuplasty,” issued Nov. 5, 2019, is incorporated by reference herein, in its entirety. In the event that the operator is also using an endoscope, the endoscopic image may also be provided on the display screen 56.

The images provided through the display screen 56 may help guide the operator in maneuvering and otherwise manipulating instruments within the patient's head (H). It should also be understood that other components of a surgical instrument and other kinds of surgical instruments, as described below, may incorporate a navigation sensor like the navigation sensor described above.

### II. Example of Suction Instrument with Deformable Multi-Piece Grip Assembly

Various surgical procedures may warrant the use of a suction instrument in order to clear fluids and/or debris from the surgical field and/or from other sites within a patient. For instance, suction may be desirable in FESS procedures, sinuplasty procedures, and/or in various other ENT procedures. In some instances during a surgical procedure, debris and/or fluids cleared may inadvertently block, clog, or otherwise inhibit fluid communication from a portion of the suction instrument and the suction source. In such instances, suction may not be suitably communicated at the open distal tip of a suction instrument. Therefore, it may be desirable to provide quick and easy access to interior portions of the suction instrument where undesirable fluid communication blockages are prone to occur. Further, it may be desirable to provide an intuitive and easy means of allowing a user of the surgical instrument to control the amount of suction being provided at the open distal tip.

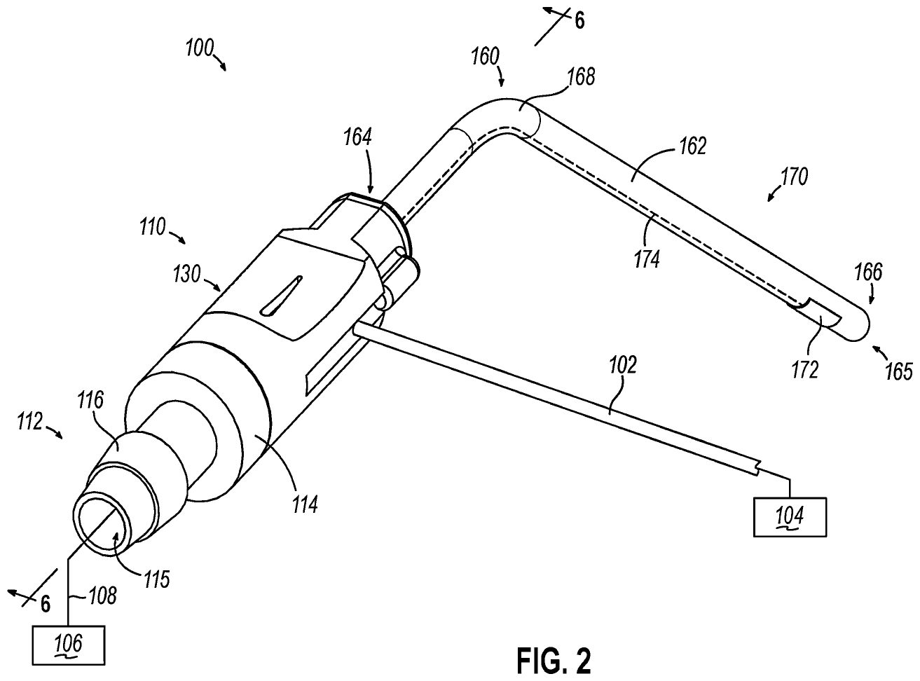

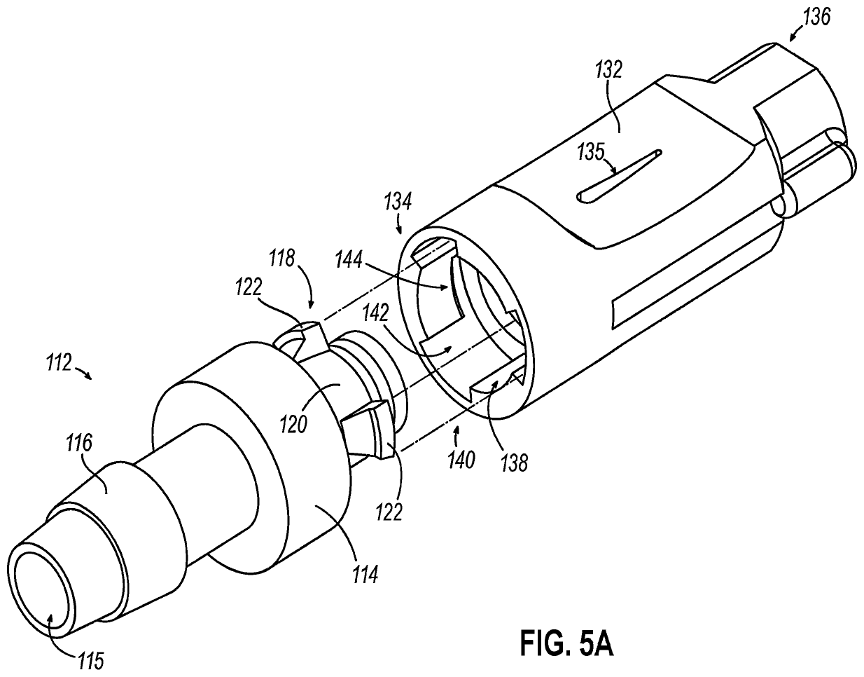

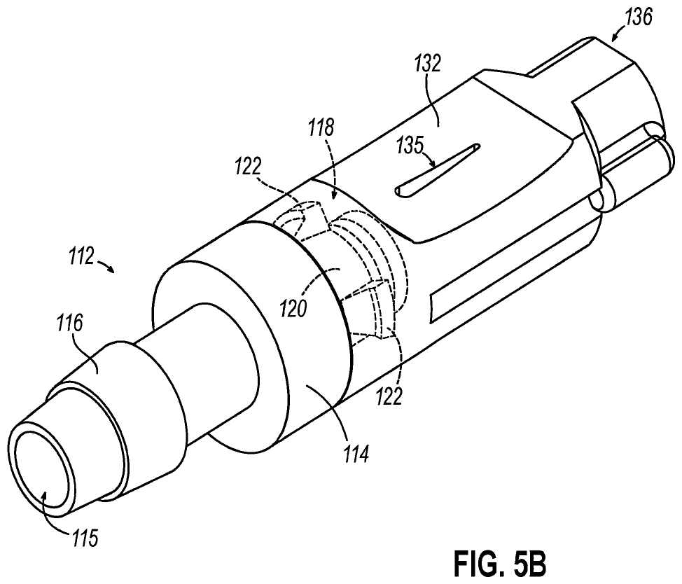

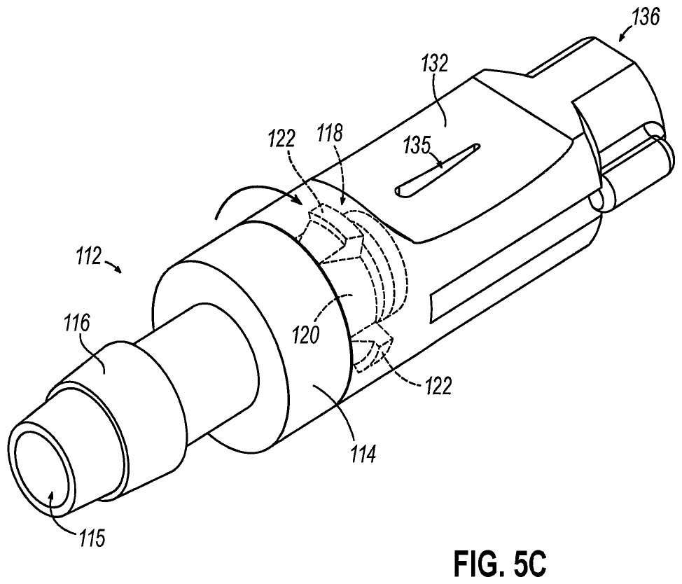

FIG. 2 shows an example of a suction instrument 100 operable to provide suction during surgical procedures, and which is configured for use with The IGS navigation system 50 described above. The suction instrument 100 includes a handle assembly 110, a shaft assembly 160 extending distally from the handle assembly 110, and a sensor assembly 170. As will be described in greater detail below, the handle assembly 110 includes a grasping portion 130 and a removable fluid coupling body 112 configured to selectively decouple (see FIG. 5A) with each other in order to provide quick and easy access to the interior of the handle assembly 110 and/or the proximal end 164 of the shaft assembly 160 to clear any undesirable accumulation of fluid/debris. As will also be described in greater detail below, the grasping portion 130 is formed of an elastically deformable material that an operator may compress, expand, and/or otherwise deform in order to control the amount of suction provided at an open distal tip 166 of the shaft assembly 160.

The shaft assembly 160 includes an elongate cannula body 162 extending from the proximal end 164 to the open distal tip 166, with an intermediate bend 168 interposed therebetween. The cannula body 162 defines a lumen 165 extending from the open distal tip 166 to the proximal end 164. In the current example, the cannula body 162 is substantially rigid such that the distal end of the cannula body 162 (including the open distal tip 166) is configured to be inserted, transnasally or otherwise, within or adjacent to a nasal cavity of a patient (or elsewhere within a patient) to provide suction at a selected surgical site via the open distal tip 166 and the lumen 165. During an example of use, when the handle assembly 110 is fully assembled, the lumen 165 of the cannula body 162 is in fluid communication with a suitable suction source 106 via pathways defined by the handle assembly 110 and the fluid conduit 108 such that the open distal tip 166 is operable to provide suction during surgical procedures.

The sensor assembly 170 includes a navigation sensor 172, a communication line 174, a cable 102, and a connector 104. The communication line 174 extends from the navigation sensor 172, along the length of the cannula body 162, within a portion of the handle assembly 110, and into communication with the cable 102. The cable 102 extends away from the handle assembly 110 and terminates into the connector 104, which is configured to establish communication with the IGS navigation system 50 during an example of use.

The communication line 174 may include a plurality of electrical communication paths extending through the cannula body 162, into the handle assembly 110, and in communication with the cable 102. Such electrical communication paths may take on a variety of forms. For instance, in some examples, such electrical communication paths may be configured as traces on a flexible printed circuit board or flex circuit attached to (e.g., on the inner diameter or outer diameter) or embedded within a portion of the cannula body 162. In other examples, such electrical communication paths may be configured as a plurality of wires extending through and/or along the cannula body 162. Such wires may likewise be attached to or embedded within a portion of the cannula body 162. Alternatively, in some examples, the cannula body 162 may be configured to define one or more dedicated lumens configured to receive one or more of such wires. Of course, various other means of facilitating electrical communication paths through the cannula body 162 and/or the handle assembly 110 will be apparent to those of ordinary skill in the art in view of the teachings herein.

The navigation sensor 172 is attached to the cannula body 162 at a location proximal to the open distal tip 166. The navigation sensor 172 is configured to provide a signal indicative of its position in response to the alternating magnetic field generated by the field generators 64 of the IGS navigation system 50. The navigation sensor 172 is operable to communicate signals to the console 20 of the IGS navigation system 50 via the communication line 174, the cable 102, and the connector 104. Based on these signals, and the known spatial relationship of the navigation sensor 172 relative to the open distal tip 166, the processor 18 may execute an algorithm to determine a three-dimensional spatial location of the distal end of the cannula assembly 160 relative to the anatomy of patient (P). While one sensor 172 is used in the current example, any suitable number of the navigation sensors 172 may be utilized as would be apparent to one skilled in the art in view of the teachings herein. Such sensors 172 may be located at suitable locations along the cannula body 162 and/or other suitable component of the instrument 100 as would be apparent to one skilled in the art in view of the teachings herein.

As mentioned above, the handle assembly 110 includes the removable fluid coupling body 112 and the grasping portion 130 configured to selectively couple and decouple from each other in order to provide quick and easy access to interior portions therefore, as well as the lumen 165 at the proximal end 164 of the cannula body 162.

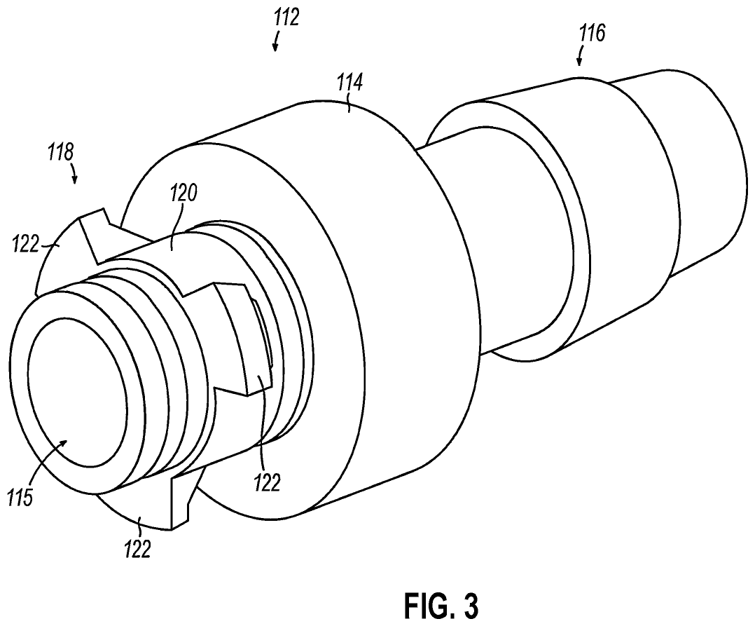

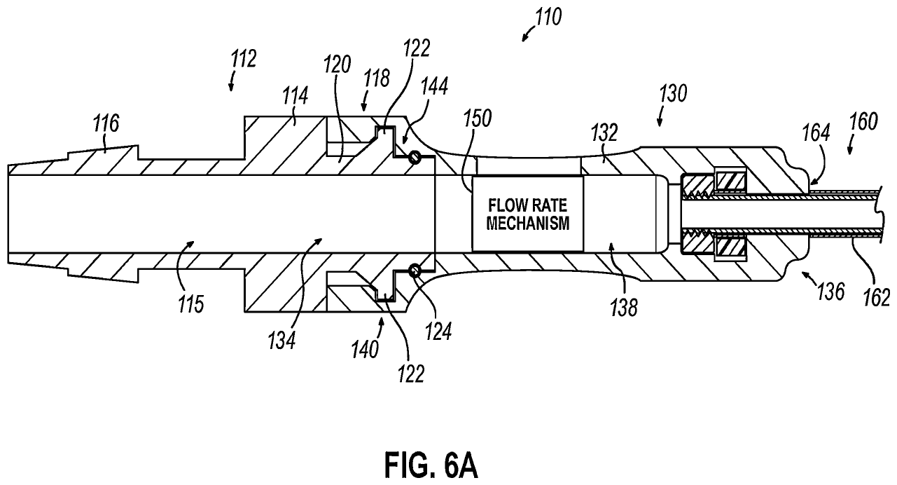

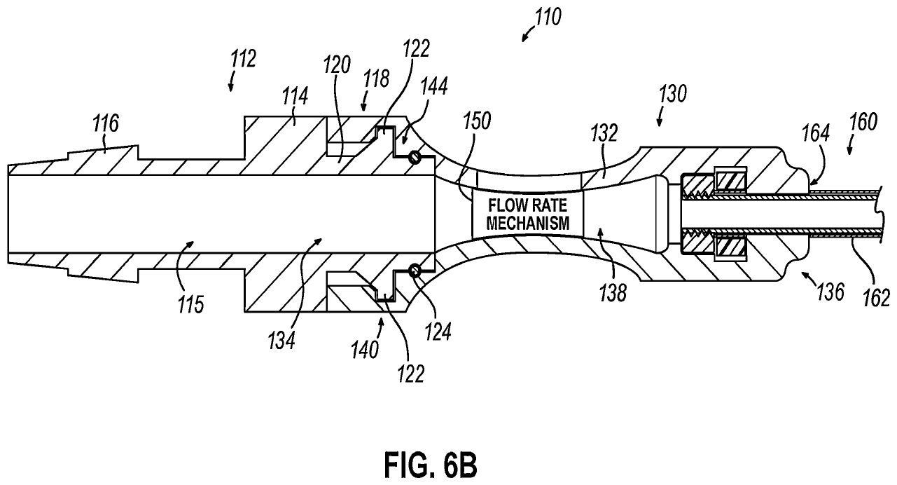

Turning to FIG. 3, the fluid coupling body 112 includes a grip 114 interposed between a proximal fluid source coupling 116 and a distal handle attachment coupling 118. When attaching and detaching the fluid coupling body 112 from the grasping portion 130 in accordance with the description herein, a user may control the fluid coupling body 112 by grasping the grip 114 and/or the proximal fluid source coupling 116. The removable fluid coupling body 112 defines a lumen 115 that extends along the length of the body 112 between respective ends of the fluid source coupling 116 and the distal handle attachment coupling 118. The proximal fluid source coupling 116 is configured to establish fluid communication with a suction source 106 via a fluid conduit 108. The lumen 115 is dimensioned to provide fluid communication between the fluid conduit 108 and a fluid communication chamber 138 (see FIGS. 5A, and 6A-6B) when the fluid coupling body 112 is suitably attached to the grasping portion 130 in accordance with the description herein.

The proximal fluid source coupling 116 may include any suitable structures as would be apparent to one skilled in the art in view of the teachings herein. For example, the proximal fluid source coupling 116 may include barbed features, a luer fitting, etc. The suction source 106 may include a vacuum pump and a fluid reservoir, among other components readily apparent to persons of ordinary skill in the art, and is configured to provide enough suction at a surgical site to pull excess fluid and/or debris proximally through the suction instrument 100.

The distal handle attachment coupling 118 includes an annular base 120 and a plurality of projections 122 extending radially away from the annular base 120. As shown in FIGS. 5A-6B, the annular base 120 and the radial projections 122 are dimensioned to fit within a proximal opening 134 defined by the grasping portion 130 in order to suitably attach the fluid coupling body 112 to the grasping portion 130 in accordance with the description herein. As best shown in FIGS. 6A-6B, when the grasping portion 130 and the fluid coupling body 112 are suitably attached, the annular base 120 is dimension to abut against a fluid seal 124 (e.g., an O-ring) disposed within the fluid communication chamber 138 defined by the grasping portion 130, thereby promoting suitable fluid communication between the lumen 115 and the fluid communication chamber 138 when suitably attached.

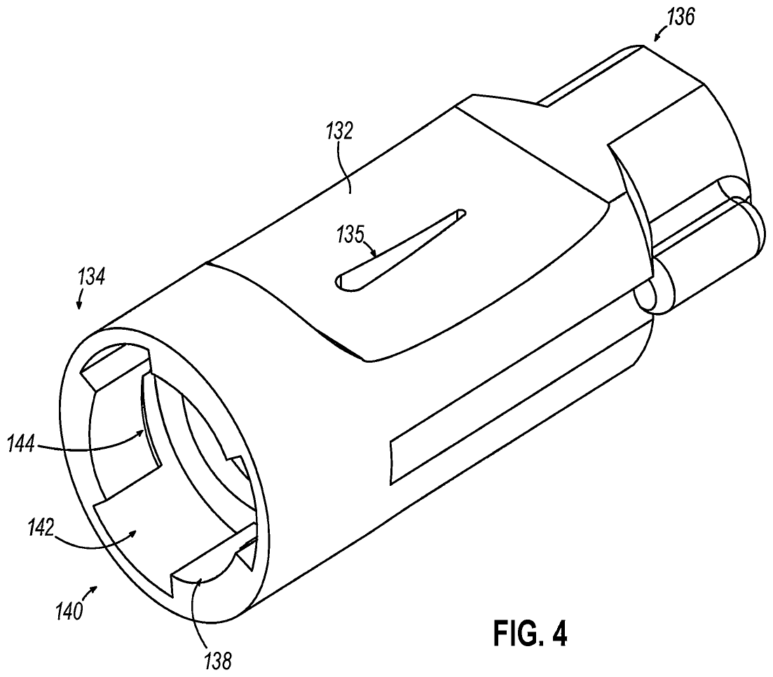

The projections 122 of the distal handle attachment coupling 118 are configured to form a bayonet fitting with a proximal coupling end 140 (see FIGS. 4-5A) of the grasping portion 130, thereby allowing the removable fluid coupling body 112 to be selectively detached from (and reattached to) the grasping portion 130. While three substantially equiangularly spaced apart projections 122 are shown, this is merely optional. Any suitable number of projections 122 with any suitably geometry may be used as would be apparent to one skilled in the art in view of the teachings herein. In some instances, the projections 122 may be arranged such that the distal handle assembly coupling 118 may be inserted within the proximal opening 134 of the grasping portion 130 at a single orientation.

Turning to FIG. 4, the grasping portion 130 includes a deformable body 132 extending between a proximal opening 134 and a distal end 136. The deformable body 132 defines a fluid communication chamber 138 that extends between the proximal opening 134 and the distal end 136. As mentioned above, the fluid communication chamber 138 is in fluid communication with the lumen 115 of the removable fluid coupling body 112 when the grasping portion 130 and the removable fluid coupling body 112 are suitably attached (see FIGS. 6A-6B). Additionally, the distal end 136 of the grasping portion 130 is attached to the proximal end 164 of the shaft assembly 160 such that the fluid communication of the cannula body 162. Therefore, when the handle assembly chamber 138 is also in fluid communication with the lumen 165 110 is suitably attached, suction may be communicated from the suction source 106 to the open distal tip 166 of the shaft assembly 160 via the lumen 165 of the cannula body 162, the fluid communication chamber 138 of the grasping portion 130, the lumen 115 of the removable fluid coupling body 112, and the fluid conduit 108. Any suitable structures may be used to attach the proximal end 164 of the shaft assembly 160 with the distal end 136 of the grasping portion 130 as would be apparent to one skilled in the art in view of the teachings herein.

In the current example, an outer surface of the deformable body 132 defines a suction control opening 135. A user may place their finger over all or portions of the suction control opening 135 in order to control the amount of suction transmitted to the open distal tip 166 of the shaft assembly 160.

As mentioned above, the projections 122 (see FIG. 3) of the distal handle attachment coupling 118 are configured to form a bayonet fitting with the proximal coupling end 140. The proximal coupling end 140 defines a plurality of access recesses 142 in communication with a respective housing recess 144. The access recesses 142 extend from a proximal end of the grasping portion 130 distally into the chamber 138. Each housing recess 144 extends circumferentially from a distal portion of a respective housing recess 144. Therefore, while the housing recess 144 is in commutation with its respective access recess 142, the recesses 142, 144 are angularly offset from each other.

As shown between FIGS. 5A-5B, the access recesses 142 are dimensioned to initially receive a portion of a respective projection 122. Therefore, if a user desires to initially couple the grasping portion 130 with the removable fluid coupling body 112, the user may align projections 122 with the access recess 142, as shown in FIG. 5A, and then actuate the two components toward each other until projections 122 are received within a respective access recess 142 and longitudinally aligned with a respective housing recess 144.

Next, as shown between FIGS. 5B-5C, once the projections 122 are longitudinally aligned with their respective housing recesses 144, the user may rotate the grasping portion 130 and the removable fluid coupling body 112 relative to each other in an angular direction such that the projections 122 rotate within the confinement and along the predefined path of the housing recess 144. As shown in FIGS. 5C-6B, with the projections 122 housed within their respective housing recesses 144, the grasping portion 130 and removal the fluid coupling body 112 are inhibited from dissociating with each other by pulling the two components away from each other.

As mentioned above, in some instances during a surgical procedure, debris and/or fluids may inadvertently block, clog, or otherwise inhibit fluid communication from a portion of the suction instrument and the suction source. If a user encounters such a blockage in suction while using the instrument 100 in accordance with the description herein, the user may decouple the removable fluid coupling body 112 from the grasping portion 130 by rotating protections 122 into alignment with a respective access recess 142 (i.e. as shown from FIG. 5C to FIG. 5B) such that the housing recesses 144 no longer engages their respective projections 122 to inhibit the removable fluid coupling body 112 from disassociating with the grasping portion 130. Next, the user may pull apart the fluid coupling body 112 and the grasping portion 130 such that the projections 122 actuate out of their respective access recess 142, thereby decoupling the removable fluid coupling body 112 from the grasping portion 130.

With the fluid communication chamber 138 and the lumen 115 exposed, a user may visually inspect the interior sections of the handle assembly 110, along with a proximal portion of the elongate tubular body 162 to determine if any obstructions are present. Next, the user may remove the obstructions and recouple the removable fluid coupling body 112 with the grasping portion 130, in accordance with the teaching herein. Therefore, it should be understood that the handle assembly 110 provides for an ability to remove unwanted debris obstructing fluid communication between the suction source 106 and the open distal tip 166 of the shaft assembly 160 during a surgical procedure.

While the removable fluid coupling body 112 and the grasping portion 130 are configured to selectively attach with one another via a bayonet fitting, any other suitable means for selectively attaching the fluid coupling body 112 and the grasping portion 130 may be utilized as would be apparent to one skilled in the art in view of the teachings herein. For example, resilient latches may be used to selectively couple the fluid coupling body 112 and the grasping portion 130.

As mentioned above, the deformable body 132 may be compressed, expanded, or otherwise deformed by a user while the open distal tip 166 is applying suction at a suitable location during an example of use. Deformation of the body 132 may alter the geometry of the fluid communication chamber 138, which in turn may alter the amount of suction communicated to the open distal tip 166 during an example of use. Therefore, as shown between FIGS. 6A-6B, a user may control the amount of suction at the open distal tip 166 by deforming the body 132. In some instances, compressing the deformable body 132 from the position shown in FIG. 6A to the position shown in FIG. 6B may be configured to reduce the amount of suction at the open distal tip 166. Conversely, compressing the deformable body 132 from the position shown in FIG. 6A to the position shown in FIG. 6B may be configured to increase the amount of suction at the open distal tip 166. The deformable body 132 may be resiliently biased toward the position shown in FIG. 6A, such that after a user no longer compresses the body 132 in the state shown in FIG. 6B, the deformable body 132 returns to the position shown in FIG. 6A. The deformable body 132 may be formed of any suitable material as would be apparent to one skilled in the art in view of the teachings herein.

As also shown in FIGS. 6A-6B, in some examples, one or more flow rate mechanisms 150 may be housed within the fluid communication chamber 138. The flow rate mechanism 150 may be strategically positioned within the fluid communication chamber 138 in order to achieve the desired changes in suction provided at the distal tip 166 in response to deformation of the deformable body 132. For example, in some instances, the flow rate mechanism 150 may be configured to allow for maximum suction when expanded as shown in FIG. 6A, allow for minimum suction when compressed as shown in FIG. 6B, and allow for a progressive change in suction when compressed in positions between those shown in FIGS. 6A-6B. As another example, in some instances, the flow rate mechanism 150 may be configured to allow for minimum suction when expanded as shown in FIG. 6A, allow for maximum suction when compressed as shown in FIG. 6B, and allow for a progressive change in suction when compressed in positions between those shown in FIGS. 6A-6B. In some instances, the flow rate mechanism 150 is compressible and resiliently biased toward a natural position. In other instances, the flow rate mechanism 150 is compressible, but not resiliently biased toward any position. Yet in other instances, the flow rate mechanism 150 is not compressible, but compression of the deformable body 132 moves the flow rate mechanism 150 into a position to achieve its intended functionality (i.e., altering the amount of suction communicated to the open distal tip 166).

### III. Example of Suction Instrument with Reusable and Disposable Components

In some instances, it may be desirable to have a section instrument including a combination of reusable components and disposable components, where the reusable components may be sterilized between uses, and the disposable components may be discarded or otherwise reprocessed after a single use (or a predetermined amount of uses).

In some instances, where a suction instrument is configured to be used in conjunction with the IGS navigation system 50, it may be desirable to have a suction instrument with interchangeable shaft assemblies that may each operatively couple with a common support structure as well as the IGS navigation system 50. In such instances, it may be desirable to store a calibration profile of a specific interchangeable shaft assembly on electronics associated with the interchangeable shaft assembly. A calibration profile for a specific interchangeable shaft assembly may include, but is not limited to, the shape and bend angle of the specific shaft assembly, the distance between an open distal tip and a navigation sensor of the interchangeable shaft assembly, the sensitivity of the navigation sensor(s) of the interchangeable shaft assembly, and other suitable data, as would be apparent to one skilled in the art in view of the teachings herein.

Storing a calibration profile on electronics housed within the interchangeable shaft assembly may allow for a reusable cable configured to selectively provide communication between a sensor assembly of the interchangeable shaft assembly and the navigation system 50. Such a reusable cable may be operable to transmit sensor signal without being required to store calibration data of the shaft assembly being used, thereby permitting use of multiple interchangeable shaft assemblies with the IGS navigation system 50 without the need of including an interchangeable cable.

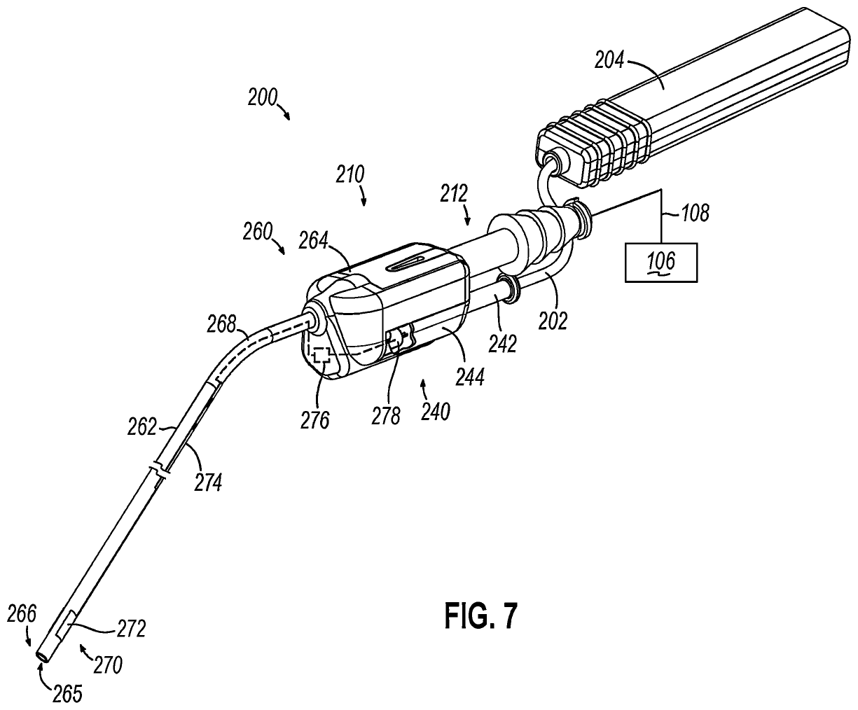

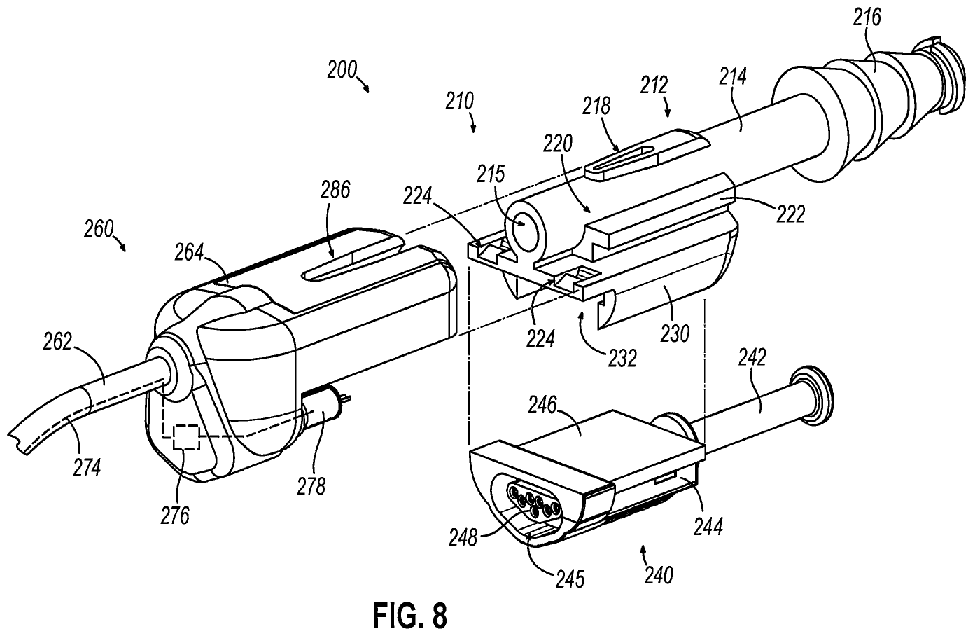

FIGS. 7-8 show an example of a suction instrument 200 operable to provide suction during surgical procedures, and which is configured for use with the IGS system 50 described above. The suction instrument 200 of the current example includes a reusable fluid coupling body 212, a reusable cable 240 configured to establish communication with the IGS navigation system 50, and a disposable shaft assembly 260. As will be described in greater detail below, the reusable fluid coupling body 212, the reusable cable 240, and the disposable shaft assembly 260 are configured to assemble together in order to form a handle assembly 210 with an elongate cannula body 262 extending distally therefrom. As will also be described in greater detail below, the disposable shaft assembly 260 includes a sensor assembly 270 configured to selectively couple with the reusable cable 240 to thereby establish communication with the IGS navigation system 50. Additionally, the sensor assembly 270 includes a programmable memory unit 276 storing a suitable calibration profile for the disposable shaft assembly 260 that may be utilized by the IGS navigation system 50 during an example of use in accordance with the description herein.

Turning to FIGS. 7-9, the disposable shaft assembly 260 includes an elongate cannula body 262, a proximal attachment body 264, and a sensor assembly 270 associated with both the elongate cannula body 262 and the proximal attachment body 264. The elongate cannula body 262 may be substantially similar to the elongate cannula body 162 described with differences herein. Therefore, the elongate cannula body 262 includes an open distal tip 266 and an intermediate bend 268 and defines a lumen 265, which may be substantially similar to the open distal tip 166, the intermediate bend 168, and the lumen 165 described above.

The elongate cannula body 262 extends distally from the proximal attachment body 264. The lumen 265 is in fluid communication with an interior of the proximal attachment body 264. When the proximal attachment body 264 is suitably attached to the reusable fluid coupling body 212 in accordance with the description herein, the lumen 265 is in fluid communication with a lumen 215 defined by reusable fluid coupling body 212. As will be described greater detail below, the fluid coupling body 212 is configured to fluidly couple with the suction source 106 via the fluid conduit 108. Therefore, during an example of use, the suction source 106 may generate suction at the open distal tip 266 via the lumens 265, 215 and the fluid conduit 108.

The sensor assembly 270 includes a sensor 272 and the communication line 274, which may be substantially similar to the sensor 172 and the communication line 174 described above, with differences elaborated herein. Instead of the communication line 274 being directly coupled to a cable and a connector configured to establish communication with the IGS navigation system 50, the communication line 274 operatively extends between the sensor 272 and the programmable memory unit 276 that is housed within the proximal attachment body 264. The communication line 274 is also in communication with a male connector 278 configured to suitably mate with a female connector 248 of the reusable cable 240. When suitably coupled, connectors 278, 248 allow information from the sensor 272 and/or the programmable memory unit 276 to be transferred to the IGS navigation system 50 during an example of use in accordance with the described herein. While in the current example, the connector 278 of the sensor assembly 270 is a male connector and the connector 248 is a female connector, this is merely optional. In some instances, the connector 278 is the female connector and the connector 248 of the reusable cable 240 is the male connector. Of course, the connectors 248, 278 may take on any other suitable structures as would be apparent to one skilled in the art in view of the teachings herein.

As mentioned above, the programmable memory unit 276 stores a suitable calibration profile of the disposable shaft assembly 260, which may include the geometric information (e.g., shape, bend angle, dimensions) of the elongate cannula body 262, specific data related to the sensor(s) 272, etc. The calibration profile stored on the programmable memory unit 276 may be communicated to the IGS navigation system 50 in accordance with the description herein such that the IGS navigation system 50 may suitably utilize data provided by the sensor 272 during an example of use in accordance with the description herein.

With the programmable memory unit 276 storing data related to a specific the disposable shaft assembly 260, a plurality of disposable shaft assemblies 260 having varying geometric features and/or sensors 272 may be readily attached to the reusable fluid coupling body 212 and the reusable cable 240 in order to function as the suction instrument 200 while still being operable for use in conjunction with the IGS navigation system 50. Further, with calibration data stored on the memory unit 276, the reusable cable 240 may not be required to store such data. Therefore, the reusable cable 240 may be operable to couple with a variety of different disposable shaft assemblies 260 having a variety of profiles and configurations. This may simplify the components required for the reusable cable 240 to function as a communication intermediary between the IGS navigation system 50 and the disposable shaft assembly 260.

The programmable memory unit 276 may have any suitable structure/features as would be apparent to one skilled in the art in view of the teachings herein. For example, the programmable memory unit 276 may include electrically erasable programmable read-only memory (EEPROM). Additionally, or alternatively, the programmable memory unit 276 may include a PCB board. In instances where EEPROM and PCB board are present, such components may be in communication with each other.

As mentioned above, the elongate cannula body 262 extends distally from the proximal attachment body 264. The proximal attachment body 264 is configured to selectively attach to both the reusable fluid coupling body 212 and the reusable cable 240 in order to form the handle assembly 210. Therefore, the proximal attachment body 264 acts as a mechanical support for the elongate cannula body 262. The proximal attachment body 264 may be formed of any suitable material using any suitable technique as would be apparent to one skilled in the art in view of the teachings herein. For example, the proximal attachment body 263 may be formed with injection molded plastic.

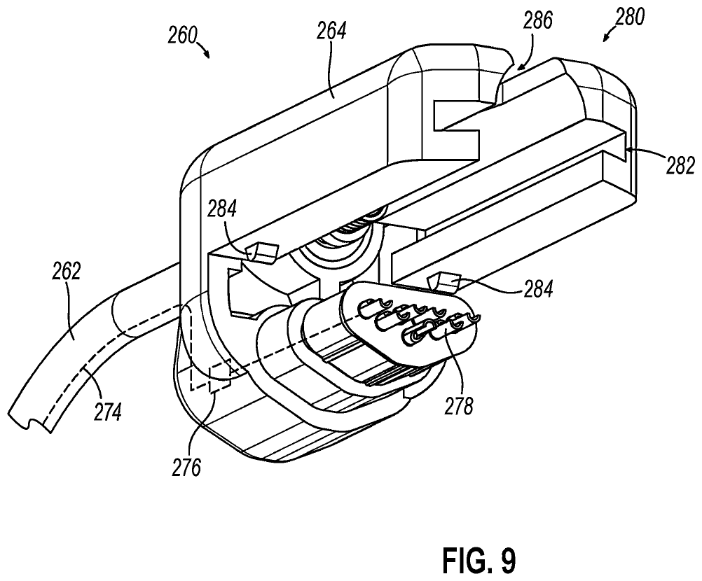

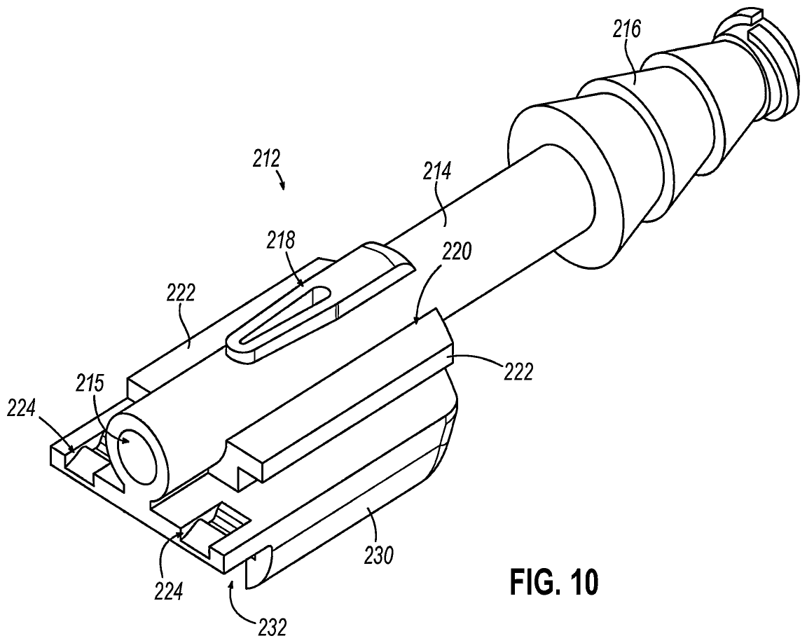

As shown in FIGS. 8-9, a proximal end of the proximal attachment body 264 defines a pair of receiving slots 282 in communication with a longitudinal slot 286. A lower portion of the longitudinal slot 286 is dimensioned to house a fluid communication body 214 (see FIG. 10) of the reusable fluid coupling body 212. A scaling element (e.g., an O-ring) may be disposed within the longitudinal slot 286 and configured to provide a fluid tight seal between the lumen 215 of the fluid communication body 214 and the lumen 265 of the elongate cannula body 262 when suitably coupled. A top portion of the longitudinal slot 286 is dimensioned to house a portion of the fluid coupling body 212 defining a section control opening 218.

The receiving slots 282 are dimensioned to receive the lateral projections 222 (see FIG. 10) of the reusable fluid coupling assembly 212 such that the reusable fluid coupling assembly 212 is laterally and rotationally constrained relative to the proximal attachment body 264 via engagement between the receiving slots 282 and the lateral projections 222. An underside of the proximal attachment body 264 includes at least one snap-fit projection 284. Each of the snap-fit projections 284 is configured to engage a snap-fit feature 224 (see FIG. 10) of the reusable fluid coupling body 212 once fully inserted into the longitudinal slot 286. Once suitably coupled to each other, engagement between the snap-fit projection 284 and the snap-fit features 224 inhibits the reusable fluid coupling body 212 and the reusable cable 240 from inadvertently disassociating with the disposable shaft assembly 260 during example use. When a user desires to decouple the reusable fluid coupling body 212 and the reusable cable 240 from the disposable shaft assembly 260, a user may pull the reusable fluid coupling body 212 and the proximal attachment body 264 apart with sufficient force to overcome the force holding the two elements together (e.g., the interaction between the snap-fit features 224 and the projections 284). Therefore, the snap-fit features 224 and the projections 284 allow for the assembly and an example of use of the handle assembly 210, as well as the disassembly of the handle assembly 210 after an example of usc.

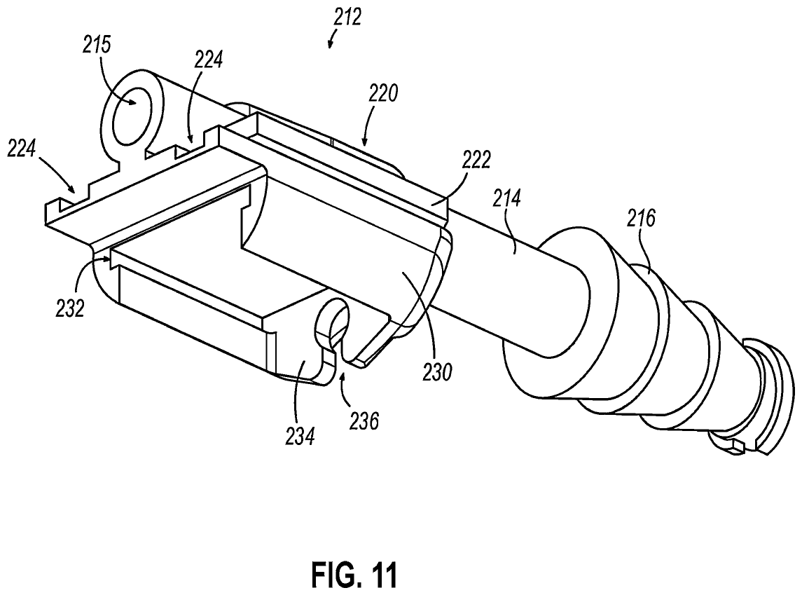

FIGS. 10-11 show the reusable fluid coupling body 212. The reusable fluid coupling body 212 is configured to fluidly couple to the suction source 106 with a fluid conduit 108 in a substantially similar manner as the fluid coupling body 112 described above. Therefore, the fluid coupling body 212 includes a proximal fluid source coupling 216 that is substantially similar to the proximal fluid source coupling 116 described above, with differences elaborated herein. However, since the reusable fluid coupling body 212 is intended to be reused for multiple procedures, the reusable fluid coupling body 212 is formed of a suitable material that is robust enough to withstand multiple sterilization processes. As one example, the reusable fluid coupling body 212 is formed of stainless steel.

As mentioned above, the reusable fluid coupling body 212 includes a fluid communication body 214 defining a lumen 215 that extends between a distal end and the proximal fluid source coupling 216. The suction control opening 218 provides communication between the lumen 215 and an exterior of the fluid communication body 214. The suction control opening 218 thereby functions in a substantially similar manner to the suction control opening 135 described above. As also mentioned above, the reusable fluid coupling body 212 also includes the lateral projections 222 and the snap-fit features 224, which are configured to allow the reusable fluid coupling body 212 to selectively attach to the proximal attachment body 264 of the disposable shaft assembly 260.

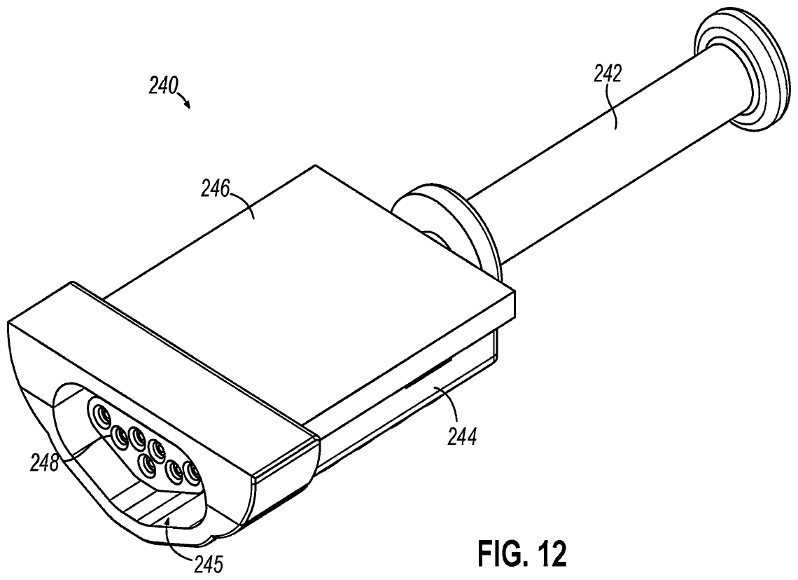

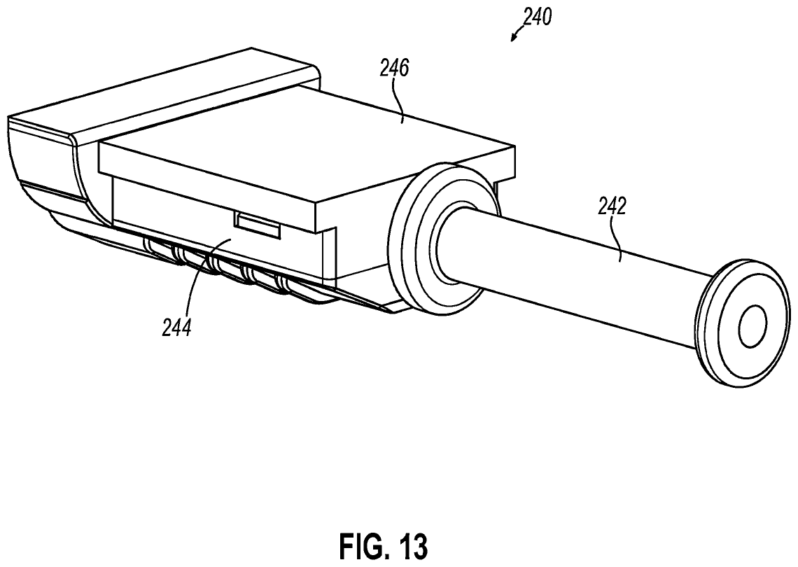

The reusable fluid coupling body 212 also includes a cable docking station 230 configured to selectively receive the reusable cable 240 (see FIGS. 12-13) such that the reusable cable 240 and the reusable fluid coupling body 212 may attach to the disposable shaft assembly 260 as a unit. The cable docking station 230 defines a central housing channel extending from a distal end all the way to a proximal stopping surface 234. The cable docking station 230 also defines a pair of receiving slots 232 dimensioned to house a respective lateral coupling body 246 (see FIGS. 12-13) of the reusable cable 240, such that the reusable cable 240 is vertically supported within the cable docking station 230 and the reusable cable 240 is laterally and rotationally constrained relative to the cable docking station 230 via engagement between the receiving slots 232 and the lateral coupling bodies 246. The proximal stopping surface 234 also defines a cable receiving opening configured to receive and support a cable connector 242 (see FIGS. 12-13) of the reusable cable 240. Therefore, while a portion of the reusable cable 240 is housed within the cable docking station 230, the cable connector 242 may extend proximally away from the cable docking station 230 to establish communication with the IGS navigation system 50.

Turning to FIGS. 12-13, the reusable cable 240 includes the cable connector 242, a grasping body 244, a pair of the lateral coupling bodies 246, and a female connector 248 disposed within a recess 245 defined by the grasping body 244. The cable connector 242 may be attached to a proximally extending cable 202, which may terminate into a connector 204. The cable connector 242 is also in communication with the female connector 248, which is configured to selectively establish communication with the male connectors 278 of the disposable shaft assembly 260. Therefore, when the female connectors 248 are suitably coupled with the male connectors 278 of the disposable shaft assembly 260, the navigation sensor 272 and the programmable memory unit 276 are configured to communicate with the IGS navigation system 50 during an example of use in accordance with the description herein.

In some instances, the proximally extending cable 202 may be a component of the reusable cable 240. In some instances, the connector 204 may also be a component of the reusable cable 240. The cable 202 and the connector 204 may function in a substantially similar manner to the cable 102 and the connector 104 described above, with differences elaborated herein. Therefore, the cable 202 and the connector 204 are configured to establish communication with suitable components of the IGS navigation system 50. The reusable cable 240 may be formed of suitable materials such that the reusable cable 240 is wipe down compatible, thereby allowing the reusable cable 240 to be suitably sterilized after an example of use in preparation for use in another procedure. Additionally, or alternatively, the reusable cable 240 may be formed of suitable material allowing for gamma ray sterilization or ETO, thereby allowing the reusable cable 240 to be suitably sterilized after an example of use in preparation for use in another procedure.

At least a portion of the grasping body 244 is dimensioned to be housed within the central housing channel defined by the cable docking station 230, while the lateral coupling bodies 246 are dimensioned to be housed within respective receiving slots 232.

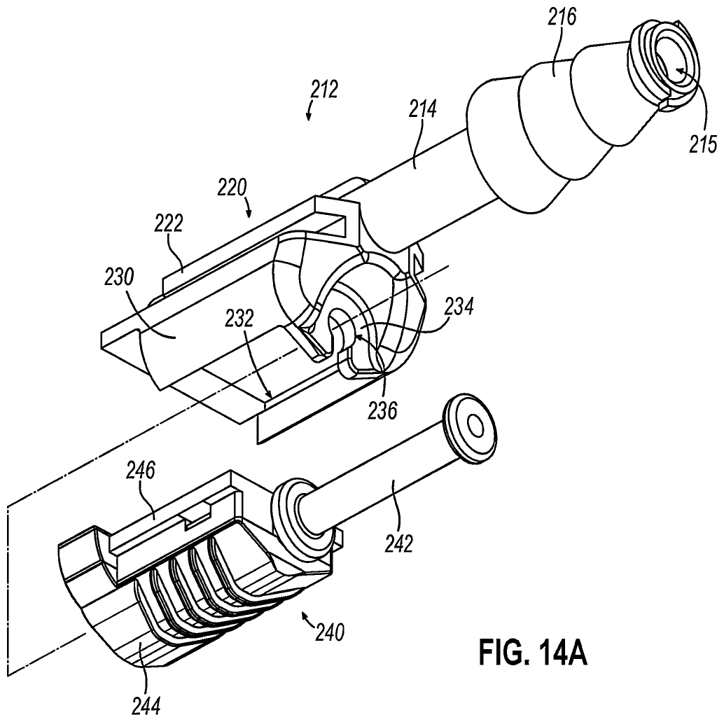

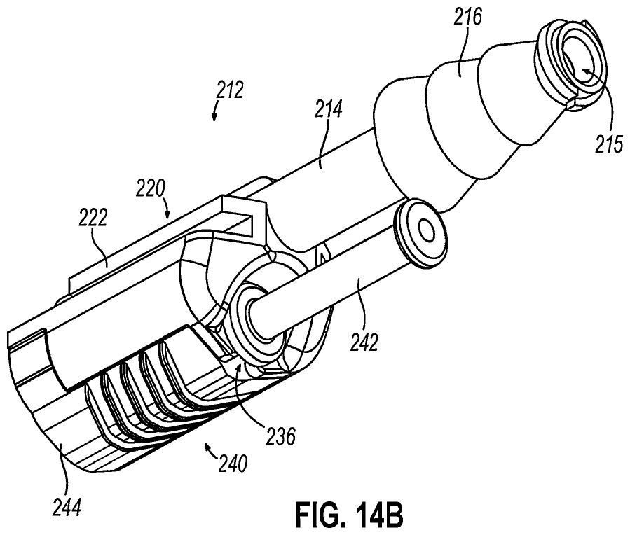

FIGS. 14A-14B show an example of a coupling of the reusable cable 240 with the reusable fluid coupling body 212. First, as shown in FIG. 14A, in order to couple the reusable cable 240 with the cable docking station 230, the lateral coupling bodies 246 may be vertically aligned with the receiving slots 232, yet distal relative to the distal end of the receiving slot 232. Next, as shown in FIG. 14B, the lateral coupling bodies 246 may then be inserted proximally into the receiving slots 232 until a proximal end of the grasping body 244 abuts against the proximal stopping surface 234 of the cable docking station 230. As the lateral coupling bodies 246 are inserted into the receiving slots 232, a portion of the grasping body 244 is also housed within the central housing channel of the cable docking station 230. Engagement between the proximal stopping surface 234 and the grasping body 244 of the reusable cable 240 may inhibit the grasping body 244 of the reusable cable 240 from being over inserted in the proximal direction.

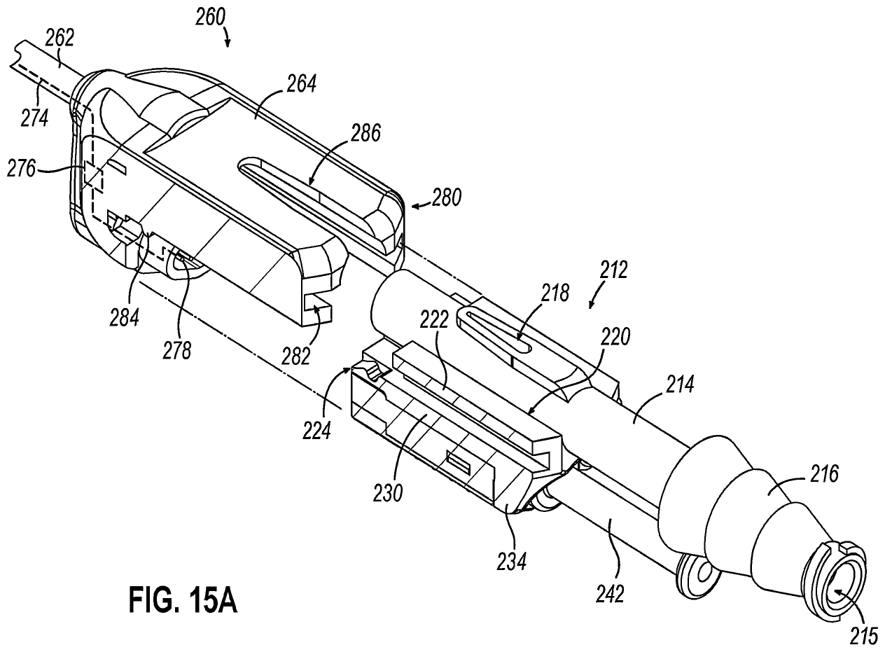

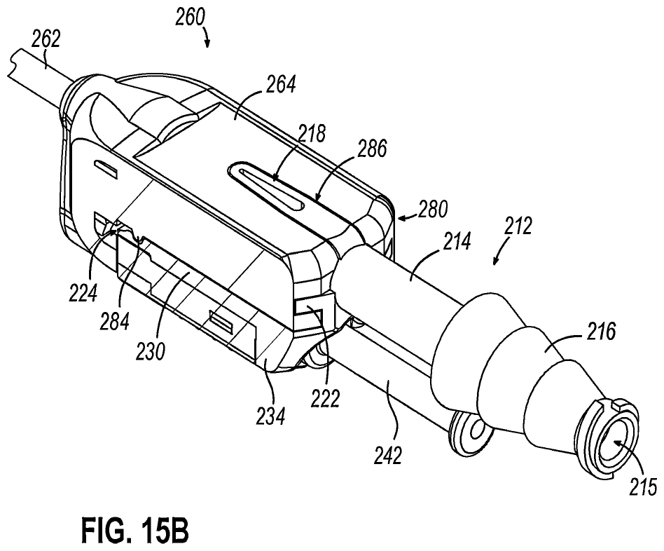

FIGS. 15A-15B show an example of a coupling of the proximal attachment body 262 of the disposable shaft assembly 260 with the recently formed unit of the reusable cable 240 and the reusable fluid coupling body 212 shown in FIG. 14B. First, as shown in FIG. 15A, a distal end of the fluid coupling body 212 is aligned with a proximal end of the proximal attachment body 264 of the disposable shaft assembly 260. In particular, the lateral projections 222 are vertically aligned with respective receiving slots 282 of the attachment body 264, yet proximal relative to a proximal end 280 of the attachment body 264.

Next, as shown in FIG. 15B, the reusable cable 240 and the fluid coupling body 212 may be actuated relative to the proximal attachment body 262 of the disposable shaft assembly 260 until the snap-fit features 224 suitably engage respective the snap-fit projections 284, thereby mechanically coupling the disposable shaft assembly 260 with the reusable fluid coupling body 212 in accordance with the description herein. Additionally, the reusable cable 240 is actuated relative to the proximal attachment body 264 until the connectors 278, 248 suitably mate with each other, thereby establishing electrical communication between the sensor assembly 270 and the reusable cable 240.

Once the handle assembly 210 is formed as shown in FIG. 15B, it should be understood that fluid communication may be transmitted from proximal fluid source coupling 216 to the open distal tip 266, thereby enabling suction to be provided at an accessed surgical site in accordance with the description herein. Additionally, electrical communication may be transmitted from both the navigation sensor 272 and the programmable memory unit 276 to the IGS navigation system 50 via the reusable cable 240 in accordance with the description herein.

### IV. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

Example 1: A suction instrument, comprising: (a) an elongate cannula comprising a proximal portion and a distal portion, the distal portion being configured to be positioned within or adjacent to an anatomical passageway of a patient, the elongate cannula defining a suction lumen in fluid communication with the distal portion, the suction lumen being configured to provide suction at the distal portion of the elongate cannula; and (b) a handle assembly associated with the proximal portion of the elongate cannula, the handle assembly comprising a deformable grasping body defining a fluid communication chamber, the fluid communication chamber being in fluid communication with the suction lumen of the elongate cannula such that the fluid communication chamber is configured to provide suction to the suction lumen, the deformable grasping body being configured to elastically deform between a relaxed state and a compressed state in order to alter the fluid communication between the fluid communication chamber and the suction lumen.

Example 2: The suction instrument of Example 1, further comprising a fluid rate mechanism configured to assist the deformable grasping body in altering the fluid communication between the fluid communication chamber and the suction lumen.

Example 3: The suction instrument of any of Examples 1 through 2, the fluid communication between the fluid communication chamber and the suction lumen being a first amount while the deformable grasping body is in the relaxed state, the fluid communication between the fluid communication chamber and the suction lumen being a second amount while the deformable grasping body is in the compressed state, the first amount being greater than the second amount.

Example 4: The suction instrument of any of Examples 1 through 2, the fluid communication between the fluid communication chamber and the suction lumen being a first amount while the deformable grasping body is in the relaxed state, the fluid communication between the fluid communication chamber and the suction lumen being a second amount while the deformable grasping body is in the compressed state, the first amount being less than the second amount.

Example 5: The suction instrument of any of Examples 1 through 4, the handle assembly further comprising a removable fluid coupling body configured to selectively attach to the deformable body.

Example 6: The suction instrument of Example 5, the deformable grasping body and the removable fluid coupling body being configured to selectively attach to each other via a bayonet fitting.

Example 7: The suction instrument of any of Examples 5 through 6, further comprising a fluid tight seal interposed between the removable fluid coupling body and the deformable grasping body.

Example 8: The suction instrument of any of Examples 1 through 7, the elongate cannula comprising an intermediate bend located between the proximal portion and the distal portion.

Example 9: The suction instrument of any of Examples 1 through 8, the elongate cannula being formed of a rigid material.

Example 10: A suction instrument, comprising: (a) an elongate cannula comprising a proximal portion and a distal portion, the distal portion being configured to be positioned within or adjacent to an anatomical passageway of a patient, the elongate cannula defining a suction lumen in fluid communication with the distal portion, the suction lumen being configured to provide suction at the distal portion of the elongate cannula; (b) a handle assembly comprising: (i) a grasping body, the proximal portion of the elongate cannula extending distally from the grasping body, the grasping body comprising an open proximal end defining a fluid communication chamber, the fluid communication chamber being in fluid communication with the suction lumen of the elongate cannula such that the fluid communication chamber is configured to provide suction to the suction lumen, (ii) a removable fluid coupling body configured to removably couple with the grasping body between an attached configuration and a detached configuration, the removable fluid coupling body, while in the attached configuration, being configured to couple the grasping body with a suction source such that the suction source may communicate suction to the distal portion of the elongate cannula, the removable fluid coupling body, while in the detached configuration, being configured to expose the fluid communication chamber at the open proximal end of the grasping body to thereby provide access to the fluid communication chamber.

Example 11: The suction instrument of Example 10, the grasping body defines a suction control opening configured to alter the amount of fluid communication between the fluid communication chamber and the suction lumen of the elongate cannula.

Example 12: The suction instrument of any of Examples 10 through 11, the grasping body is formed of a resiliently deformable material.

Example 13: The suction instrument of Example 12, the grasping body configured to deform to alter an amount of fluid communication between the fluid communication chamber and the suction lumen of the elongate cannula.

Example 14: The suction instrument of any of Examples 10 through 13, the grasping body comprising a fluid rate mechanism housed within the fluid communication chamber.

Example 15: The suction instrument of any of Examples 10 through 14, the removable fluid coupling body and the grasping body being configured to removably couple via bayonet fitting.

Example 16: The suction instrument of Example 15, the removable fluid coupling body comprising at least one projection dimensioned to be house within a bayonet slot defined by the grasping body.

Example 17: A suction instrument, comprising: (a) a shaft assembly, the shaft assembly comprising: (i) an elongate cannula comprising a proximal portion and a distal portion, the distal portion being configured to be positioned within or adjacent to an anatomical passageway of a patient, the elongate cannula defining a suction lumen in fluid communication with the distal portion, the suction lumen being configured to provide suction at the distal portion of the elongate cannula, (ii) a proximal attachment body attached to the proximal portion of the elongate cannula, the proximal attachment body defining a first receiving slot, (iii) a navigation assembly, comprising: (A) a navigation sensor, (B) a programmable memory unit being in communication with the navigation assembly, the programmable memory unit comprising a navigation sensor profile, and (C) a first connector fixed to the proximal attachment body, the first connector being in communication with both the programmable memory unit and the navigation sensor; (b) a reusable cable comprising a second connector configured to selectively couple with the first connector, the reusable cable being configured to establish communication between the navigation assembly and a navigation system; and (c) a reusable fluid coupling body configured to establish fluid communication between a suction source and the suction lumen of the elongate cannula of the shaft assembly, the reusable fluid coupling body comprising at least one protrusion dimensioned to fit within the first receiving slot of the proximal attachment body to thereby selectively attach the reusable fluid coupling body and the proximal attachment body of the shaft assembly, the reusable fluid coupling body comprising a cable docking station configured to house the reusable cable while the reusable fluid coupling body is selectively attached to proximal attachment body.

Example 18: The suction instrument of Example 17, the reusable fluid coupling body comprising a snap-fit feature, the proximal attachment body comprising a protrusion dimensioned to fit within the snap-fit feature to inhibit the reusable fluid coupling body from disassociating with the proximal attachment body.

Example 19: The suction instrument of any of Examples 17 through 18, the program memory unit further comprising an electrically erasable programmable read-only memory.

Example 20: The suction instrument of any of Examples 17 through 19, the reusable cable comprising a pair of lateral coupling bodies dimensioned to be received within the cable docking station of the reusable fluid coupling body.

Example 21: The suction instrument of any of Examples 17 through 20, the programmable memory unit being housed within the proximal attachment body.

### V. Miscellaneous

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, versions of the device may be disassembled, and any number of the particular pieces or parts of the device may be selectively replaced or removed in any combination. Upon cleaning and/or replacement of particular parts, versions of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be processed before surgery. First, a new or used instrument may be obtained and if necessary cleaned. In some instances, the instrument may be placed in a reprocessing tray (e.g., a metal bin or basket) and then cleaned in a surgical instrument washer. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a surgical facility. A device may also be sterilized using any other technique known in the art, including but not limited to beta or gamma radiation, ethylene oxide, steam, hydrogen peroxide vapor (e.g., via a STERRAD sterilization system by Advanced Sterilization Products of Irvine, California), and/or using any other suitable systems or techniques.

Having shown and described various versions of the present inventions, further adaptations of the methods and systems described herein may be accomplished by appropriate modifications by one of ordinary skill in the art without departing from the scope of the present inventions. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present inventions should be considered in terms of the following claims and is understood not to be limited to the details of structure and operation shown and described in the specification and drawings.

What is claimed is:

1. A suction instrument, comprising:
an elongate cannula comprising a proximal portion and a distal portion, wherein the distal portion is configured to be positioned within or adjacent to an anatomical passageway of a patient, and the elongate cannula defines a suction lumen in fluid communication with the distal portion, the suction lumen being configured to provide suction at the distal portion of the elongate cannula; and
a handle assembly associated with the proximal portion of the elongate cannula and comprising a deformable grasping body defining a fluid communication chamber, wherein the fluid communication chamber is in fluid communication with the suction lumen of the elongate cannula and is configured to provide suction to the suction lumen, and the deformable grasping body is configured to elastically deform between a relaxed state and a compressed state to alter the fluid communication between the fluid communication chamber and the suction lumen.

2. The suction instrument of claim 1, further comprising a fluid rate mechanism configured to assist the deformable grasping body in altering the fluid communication between the fluid communication chamber and the suction lumen.

3. The suction instrument of claim 1, wherein the fluid communication between the fluid communication chamber and the suction lumen is a first amount while the deformable grasping body is in the relaxed state, and the fluid communication between the fluid communication chamber and the suction lumen is a second amount while the deformable grasping body is in the compressed state, the first amount being greater than the second amount.

4. The suction instrument of claim 1, wherein the fluid communication between the fluid communication chamber and the suction lumen is a first amount while the deformable grasping body is in the relaxed state, and the fluid communication between the fluid communication chamber and the suction lumen is a second amount while the deformable grasping body is in the compressed state, the first amount being less than the second amount.

5. The suction instrument of claim 1, wherein the handle assembly further comprises a removable fluid coupling body configured to selectively attach to the deformable grasping body.

6. The suction instrument of claim 5, wherein the deformable grasping body and the removable fluid coupling body are configured to selectively attach to each other via a bayonet fitting.

7. The suction instrument of claim 5, further comprising a fluid tight seal interposed between the removable fluid coupling body and the deformable grasping body.

8. The suction instrument of claim 1, wherein the elongate cannula comprises an intermediate bend located between the proximal portion and the distal portion.

9. The suction instrument of claim 1, wherein the elongate cannula is formed of a rigid material.

10. A suction instrument, comprising:
an elongate cannula comprising a proximal portion and a distal portion, wherein the distal portion is configured to be positioned within or adjacent to an anatomical passageway of a patient, the elongate cannula defines a suction lumen in fluid communication with the distal portion, and the suction lumen is configured to provide suction at the distal portion of the elongate cannula; and
a handle assembly comprising:
a grasping body, wherein the proximal portion of the elongate cannula extends distally from the grasping body and the grasping body comprises an open proximal end defining a fluid communication chamber, the fluid communication chamber being in fluid communication with the suction lumen of the elongate cannula such that the fluid communication chamber is configured to provide suction to the suction lumen; and
a removable fluid coupling body configured to removably couple with the grasping body between an attached configuration and a detached configuration,
wherein, while in the attached configuration, the removable fluid coupling body is configured to couple the grasping body with a suction source such that the suction source may communicate suction to the distal portion of the elongate cannula,
wherein, while in the detached configuration, the removable fluid coupling body is configured to expose the fluid communication chamber at the open proximal end of the grasping body to thereby provide access to the fluid communication chamber.

11. The suction instrument of claim 10, wherein the grasping body defines a suction control opening configured to alter an amount of fluid communication between the fluid communication chamber and the suction lumen of the elongate cannula.

12. The suction instrument of claim 10, wherein the grasping body is formed of a resiliently deformable material.

13. The suction instrument of claim 10, wherein the grasping body is configured to deform to alter an amount of fluid communication between the fluid communication chamber and the suction lumen of the elongate cannula.

14. The suction instrument of claim 10, wherein the grasping body comprises a fluid rate mechanism housed within the fluid communication chamber.

15. The suction instrument of claim 10, wherein the removable fluid coupling body and the grasping body are configured to removably couple via bayonet fitting.

16. The suction instrument of claim 10, wherein the removable fluid coupling body comprises at least one projection dimensioned to be housed within a bayonet slot defined by the grasping body.

17. A suction instrument, comprising:
a shaft assembly comprising:
an elongate cannula comprising a proximal portion and a distal portion, wherein the distal portion is configured to be positioned within or adjacent to an anatomical passageway of a patient, and the elongate cannula defines a suction lumen in fluid communication with the distal portion, the suction lumen being configured to provide suction at the distal portion of the elongate cannula;
a proximal attachment body attached to the proximal portion of the elongate cannula, wherein the proximal attachment body defines a first receiving slot; and
a navigation assembly comprising a navigation sensor, a programmable memory unit in communication with the navigation assembly and comprising a navigation sensor profile, and a first connector fixed to the proximal attachment body, wherein the first connector is in communication with both the programmable memory unit and the navigation sensor;
a reusable cable comprising a second connector configured to selectively couple with the first connector, wherein the reusable cable is configured to establish communication between the navigation assembly and a navigation system; and
a reusable fluid coupling body configured to establish fluid communication between a suction source and the suction lumen of the elongate cannula of the shaft assembly, wherein the reusable fluid coupling body comprises (a) at least one protrusion dimensioned to fit within the first receiving slot of the proximal attachment body to thereby selectively attach the reusable fluid coupling body and the proximal attachment body of the shaft assembly and (b) a cable docking station configured to house the reusable cable while the reusable fluid coupling body is selectively attached to proximal attachment body.

18. The suction instrument of claim 17, wherein the reusable fluid coupling body comprises a snap-fit feature, and the proximal attachment body comprises a protrusion dimensioned to fit within the snap-fit feature to inhibit the reusable fluid coupling body from disassociating with the proximal attachment body.

19. The suction instrument of claim 17, wherein the reusable cable comprises a pair of lateral coupling bodies dimensioned to be received within the cable docking station of the reusable fluid coupling body.

20. The suction instrument of claim 17, wherein the programmable memory unit is housed within the proximal attachment body.
